(19) United States
(12) Patent Application Publication
(10) Pub. No.: US 2026/0077111 A1
(43) Pub. Date: Mar. 19, 2026

(54) STENT TYPE AV FLOW RESTRICTORS

(71) Applicants: Stanley Batiste, Granite Bay, CA (US); Daniel Batiste, Roseville, CA (US)

(72) Inventors: Stanley Batiste, Granite Bay, CA (US); Daniel Batiste, Roseville, CA (US)

(21) Appl. No.: 19/329,794

(22) Filed: Sep. 16, 2025

Related U.S. Application Data
(60) Provisional application No. 63/695,549, filed on Sep. 17, 2024.

(51) Int. Cl. A61M 1/36

(52) U.S. Cl. CPC A61M 1/3655

(57) ABSTRACT

A stent is provided with a central portion between two end portions, and with the central portion narrower than the end portions to provide a stenosis within the stent, such as for improving function of a dialysis graft or fistula. The stent can be merely a metal wire cage or can additionally include a liner inside and/or outside of the metal cage. One stent includes tapering transitions between the end portions and the central portion. The stent can be self-expanding with a retracted form held by a surrounding sheath until the sheath is retracted and the stent returns to its biased expanded form. A balloon can provide a radial expansion force to expand the stent. The balloon or self-expansion can expand the stent partially, with the central portion narrower than the end portions, and optionally further expanded to have a diameter matching that of the end portions.

### CROSS-REFERENCE TO RELATED APPLICATIONS

This application claims benefit under Title 35, United States Code § 119(e) of U.S. Provisional Application No. 63/695,549 filed on Sep. 17, 2024.

### FIELD OF THE INVENTION

The invention relates generally to dialysis grafts and in particular improvement in the flow and access capabilities of the dialysis grafts or fistulas through implantation of a stent with flow restriction incorporated therein.

### BACKGROUND OF THE INVENTION

There are currently more than 600,000 patients in the United States with end-stage renal disease (ESRD) and many times more than that throughout the world. ESRD accounts for approximately 6.4% of the overall Medicare budget at over $23 billion dollars in the US in 2006. Patients with end stage renal disease have lost their normal kidney function and as a result require dialysis to substitute the function of the kidney cleansing the blood. There are two types of dialysis: hemodialysis and peritoneal dialysis. For the purposes of this overview, we will primarily be focused on hemodialysis.

Hemodialysis requires that large volume blood access and exchange be consistently available to sustain the life of the patient. Typically, a dialysis patient will require 3-4 hours of dialysis three days a week. The challenge with providing hemodialysis is maintaining access to large volumes of blood when a body constantly fights attempts to keep access available by healing closed such access. Currently there are three ways to provide hemodialysis: dialysis catheters, arterial venous fistulas (AVFs) and arterial venous grafts (AVGs). Although used worldwide, catheters are known not to be efficient for long term dialysis. Unfortunately, catheters have very short patency rates and high rates of infection. For these reasons dialysis guidelines strongly oppose catheter use, other than for short term use, until fistula or graft placement is available.

AVGs and AVFs are synthetic and natural conduits respectively that are surgically placed to provide long term dialysis access. Both provide large diameter targets that can be easily accessed with large needles for blood exchange. These conduits are commonly placed in the arm with the furthest point attached to the patent's artery and then are directly attached to the vein for blood flow return. The high arterial blood pressure and flow is shunted directly to the vein providing dilatation of the vein or graft and large volume blood flow. Although these methods provide a good means of providing dialysis access, both have limitations with regard to sustaining long term patency and accessibility. The patency rates are much greater than that of a catheter however overall are relatively poor when considering the few years gained in a patient's life. It has been noted that there is only 50% shunt patency at one year and less than 25% at 2 years. Not only does this create a huge burden on the cost of healthcare but more importantly, once access is no longer available, a new access point must be created to sustain a patient's life. As far as physically accessing both need to be cannulated with large bore needles by trained technologists. Access can be very difficult.

A thorough description of the reason for dialysis fistula and graft failure is beyond the scope of this document. The fundamental problem is that the flow dynamics created by these artificial conduits are not normal to our bodies. The change is detected by the body and the normal physiologic defenses become involved and attempt to return the system to normal leading to graft or fistula failure. Failure of the graft generally means that the graft or fistula ceases to maintain flow. Once occluded the graft becomes full of blood which is static which subsequently becomes thrombus. Once failure occurs, the patient loses the ability to have hemodialysis until access function is restored.

From the discussion that follows, it will become apparent that the present invention addresses the deficiencies associated with the prior art while providing numerous additional advantages and benefits not contemplated or possible with prior art constructions.

### SUMMARY OF THE INVENTION

It has been shown that surgical improvements to failing dialysis grafts can improve long term patency and as a result increase the lifespan of patients. One successful surgical procedure involves creating a narrowing within the mid aspect of a dialysis graft or fistula. This procedure is referred to as precision banding and requires that the graft is surgically exposed, and a suture is then applied around the graft and tightened, narrowing the lumen and creating a stenosis. This stenosis (i.e. narrowing) decreases pressure, flow and pulsation improving the hemodynamic properties of the fistula or graft. Although there are currently means of creating the stenosis, this invention is unique in its construction and method of use. Two other solutions we have also invented and to which this invention relates are described in U.S. Patent Application Nos. Ser. No. 19/226,732, filed on Jun. 3, 2025 and Ser. No. 19/265,501, filed on Jul. 10, 2025, each incorporated herein by reference in their entirety.

The invention is a deployable stent flow restrictor which can be packaged in a small delivery catheter for access into a patient's vasculature. The innovation involves using a combination of both self-expanded and balloon expandable stents integrated into one device. Self-expanding stents are commonly used in the vascular and biliary system with the design allowing for instant opening once the stent is uncovered from its restrictive housing (e.g. a sheath), and expands to a larger width that it is biased to have. Balloon expandable stents are deployed in their un-open state and remain collapsed until a balloon is introduced within their center and inflated radially. To use the invention, the desired vessel is accessed using standard technique and the stent is advanced to the desired location. The outer deployment sheath is pulled back, and the self-expanding component will open and oppose the vessel wall in 360°. With further unsheathing the balloon expandable portion will then be exposed but will not expand and remain with the narrowed restrictor component. Finally, the deployment sheath will be completely removed allowing for the second self-expanding portion to open and the stent will then be free of the catheter and fully deployed in the vessel. Although the preferred configuration for desired flow restricting stent is to have the mid stenosis created by the un-inflated balloon expandable stent to remain narrowed, at some point there may be a need to fully expand the stent to allow full or partial patency of the vessel. This is then done by wire access through the stent and advancement of pre-sized balloon allowing for exact sized narrowing. The balloon is then inflated and subsequently the flow restrictor stent is further opened. The ridged walls of the stent remain open after balloon deflation. When the balloon is deflated it can then be easily removed and the stent will be left open in the lumen of the vessel.

### OBJECTS OF THE INVENTION

Accordingly, a primary object of the present invention is to provide a stent which can be percutaneously implanted from a catheter into a blood vessel, fistula, or graft, with the stent featuring a middle narrowed portion.

Another object of the present invention is to provide a stent which can be collapsed and is self-expanding when released from a sheath to have a construction with a narrowed central portion.

Another object of the present invention is to provide a stent which can be collapsed and advanced with an a catheter to an implantation site, such as within a blood vessel, fistula or graft, and then be expanded by retraction of a sheath surrounding the stent, and with the stent either being self-expanding or expanded with a balloon, and the stent featuring a narrow central section.

Another object of the present invention is to provide a stent with a narrowed central section which can be either self-expanding when a sheath is retracted therefrom or expanded by a balloon, and with the narrowed central section either resisting expansion, at least somewhat responsive to balloon expansion forces or with the narrow central section expanding to no longer be narrowed when an expansion balloon supplies sufficient expansion forces to the narrowed central section.

Another object of the present invention is to provide a method for placement of a stent which features a narrowed central section between two larger width end sections, the placement being in a blood vessel, graft or fistula and involving self-expansion and/or balloon assisted expansion.

Other further objects of the present invention will become apparent from a careful reading of the included drawing figures, the claims and detailed description of the invention.

### BRIEF DESCRIPTION OF THE DRAWINGS

FIG. 1: Shown is a dialysis machine 100 with inflow and outflow lines 15 and graft with proximal flow limb 12 and distal flow limb 10 having a mid-graft stenosis 200 hat could be from prior art “banding” or built into the graft or provided by a stent such as those of this invention.

FIG. 2: Shown are two variations of the invention. In a first variation (top of FIG. 2) 2000 is the bare metal flow restrictor stent with the two self-expanding end components “a” and the integrated balloon expandable portion “b.” In a second variation (bottom of FIG. 2), a material covered stent 3000 also has the internal (or external) metal self-expanding parts “a” and center stenosis balloon part “b.”

FIG. 3: This drawing shows, next to associated side views of FIG. 2, a cross sectional view (top) of the bare metal stent 2000 with the metal portion 2100. A cross-section of the covered stent 3000 is shown with two material configurations (bottom left) and (bottom right). A left cross-section has a single inner material covering 2250 over the metal part 2100 and right cross-section has an inner material lining 2250 and an outer covering material 2200.

FIG. 4: Three drawings show side views of the stent 2000 in its collapsed state and within a delivery catheter 2005. A pusher rod 2010 is shown within the delivery catheter abutting the back of the collapsed stent.

FIG. 5: Illustrates the mechanism of stent delivery in “c” where the stent 2000 is within the delivery catheter 2005 and the pushing rod 2010 is held in place while the delivery catheter 2005 is pulled back (or the pushing rod advances while the catheter is stationary). Images “d,” “e” and “f” show further unsheathing of the delivery catheter 2005 with first opening of the self-expanding component 2001 followed by the narrowed central section/component 2002, until the stent 2000 is fully deployed, as shown in image “f.”

FIG. 6: Illustrates intervascular stent deployment in sequential drawings “a” through “e,”into a body lumen (or graft or fistula) at a subcutaneous location.

FIG. 7: Image “a” shows wire 2500 access through the stent 2000. Image “b” shows the advancement of the un-inflated balloon 2550 over the guide wire and image “c” shows further advancement positioned inside of the stent lumen. Images “d” and “e” show balloon inflation 2550 and stent expansion, with full expansion, even of the otherwise narrowed central portion, shown in image “e.”

FIG. 8: Images “a” through “e” show sequential intervascular stent balloon dilatation while within a subcutaneous lumen.

FIG. 9: Images “a” through “e” show sequential intervascular stent balloon dilatation of the material covered stent 3000.

### DESCRIPTION OF THE PREFERRED EMBODIMENT

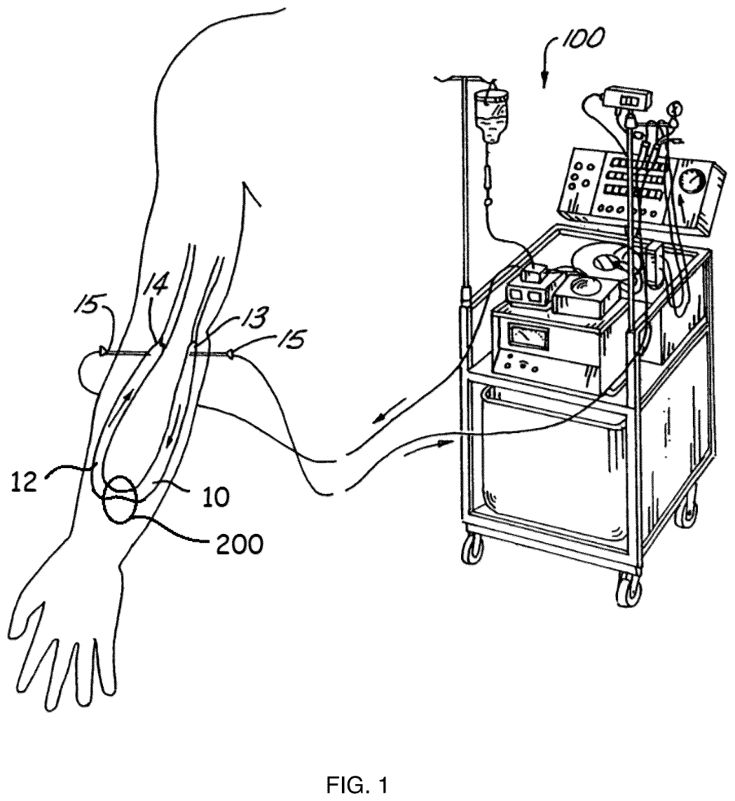

The invention is a combination of self-expanding and/or balloon expanding stents creating a flow restrictor for use in dialysis patient's fistulas and grafts. This novel construction will provide a means of deploying a stent percutaneously in order to decrease flow and pressure within the fistula or graft. FIG. 1 illustrates the attachment of the dialyzer unit (100) with the AV graft access points (15) and the mid-graft stenosis (200). High pressure from access point (15) within the proximal graft (12) benefits flow to the dialyzer (100) and low pressure distal to the mid-graft stenosis (200) in the outflow limb (10) helps low resistance flow from the dialyzer to the patient. The stenosis (200) can be provided or augmented by catheter placement of the stent 2000, 3000.

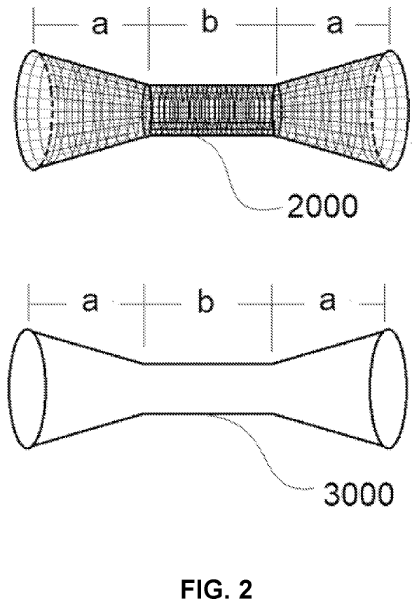

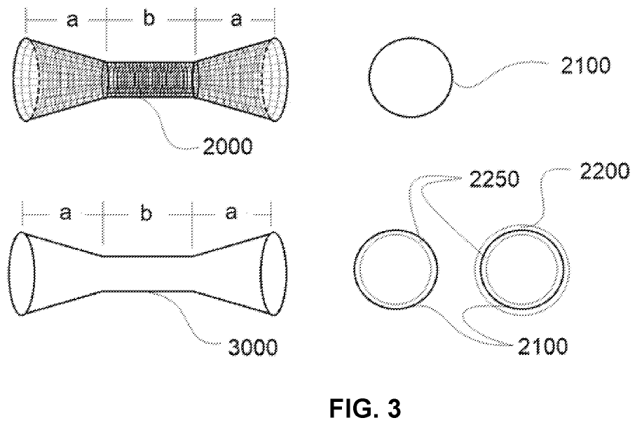

The first embodiments of the design are shown in FIG. 2 with the bare metal stent (2000) and its self-expanding end segments (a) and middle narrowed balloon expandable segment (b). The material covered stent (3000) is shown with ePTFE or other material covering the metal self-expanding segments (a) and central narrowed (but balloon expandable) segment (b) (the material either on an interior or an exterior or both). FIG. 3 illustrates side views of the bare metal stent (2000) and the covered stent (3000) with the metal components (2100) in both examples. Two sectional views (lower left and lower right) are shown of the covered stent (3000) where the inner lined portion (2250) is shown in both, and the outer covered embodiment (2200) is shown in the second (bottom left). A sectional view (top right) is also shown for the bare metal stent (2100)

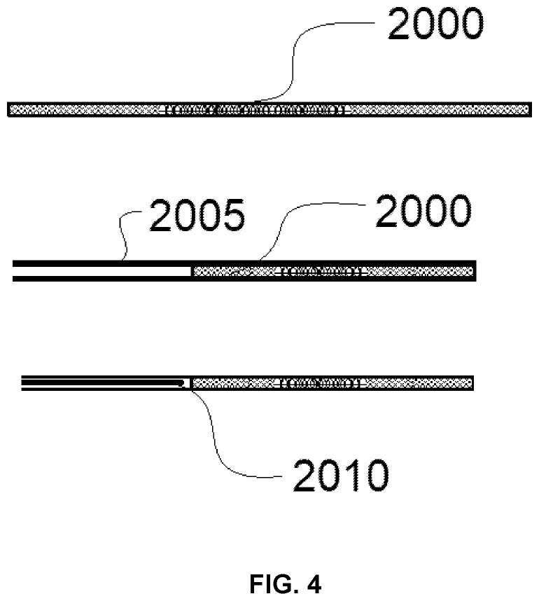

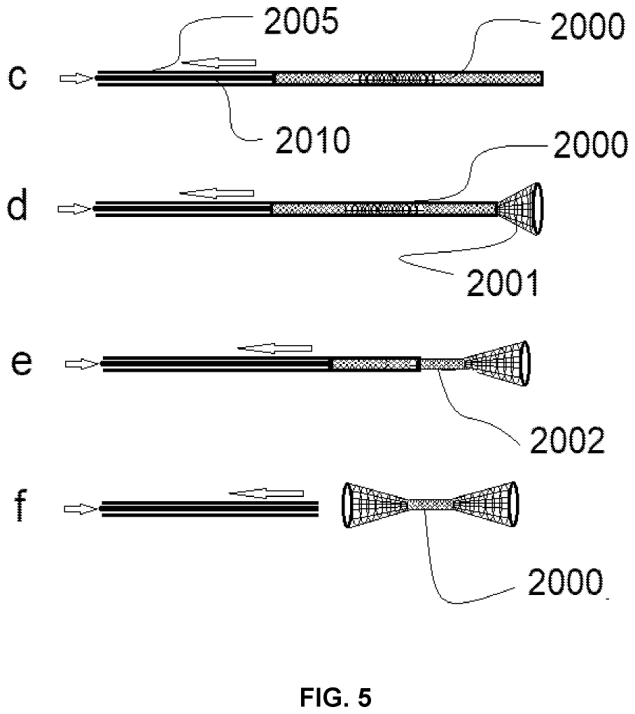

In FIG. 4 the metal stent is shown in its collapsed state (2000) and further housed within a deployment sheath (2005). The pusher rod (2010) is shown within the delivery catheter abutting the end of the stent. FIG. 5 illustrates how the stent is deployed, where image (c) shows the pusher rod (2010) held in place while the deployment sheath (2005) is retracted off of the collapsed stent (2000). Image (d) shows further retracted motion of the deployment sheath and expansion of the self-expanding portion (2001) of the stent (2000). Image (e) shows further retraction of the deployment sheath and exposure of the balloon expandable portion (2002) which remains collapsed. Image (f) shows complete deployment of the stent from the deployment sheath.

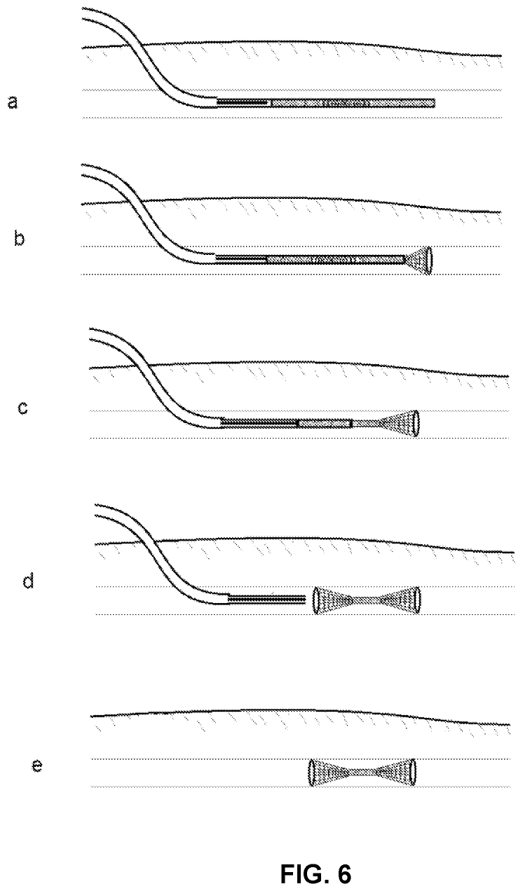

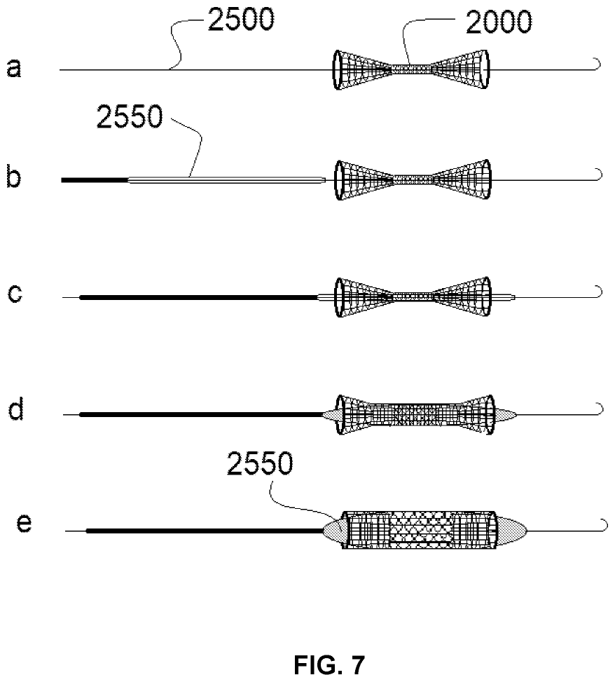

FIG. 6 shows the stent's deployment steps (images a-d) within the blood vessel, fistula or graft. Image (e) shows the stent within the desired location in the vessel (or within the graft or fistula) and removal of the deployment components. FIG. 7 illustrates the series of steps to fully expand the stent if needed. Image (a) shows guide wire (2500) access through the center of the stent. Image (b) illustrates advancement of the non-inflated balloon catheter (2550) over the guide wire and image (c) shows advancement of the balloon into position within the center of the stent. Images (d and e) show inflation of the balloon (2550) with partial expansion (d) and full expansion (e).

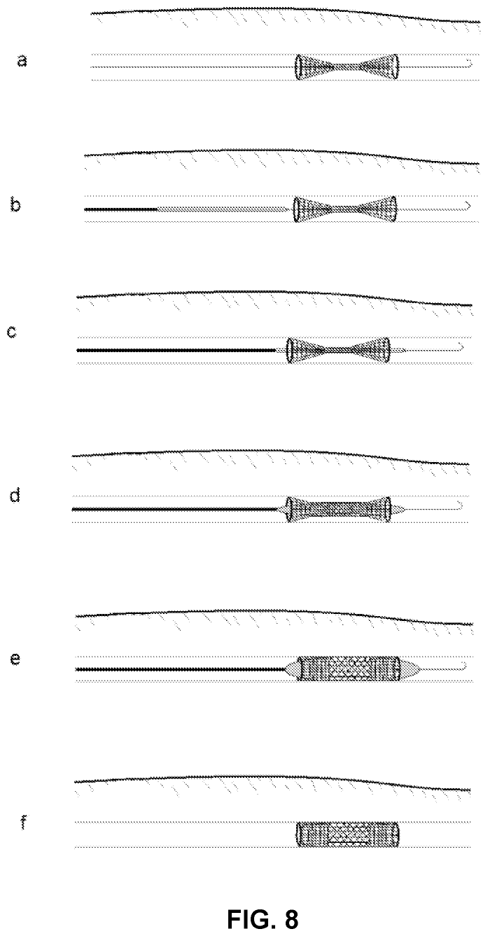

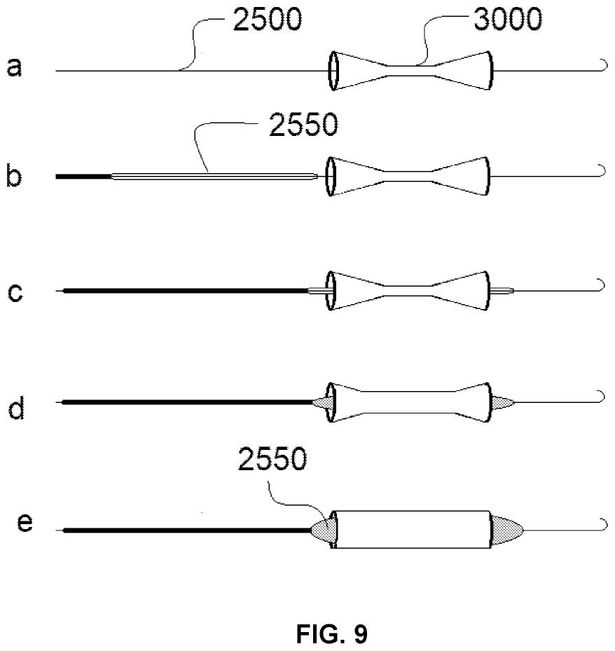

FIG. 8 shows the series of balloon inflation steps (images a-f) within the blood vessel, fistula or graft. FIG. 9 is the series of balloon inflation steps illustrated for the covered embodiment of the design. A similar technique is used where there is wire (2500) advancement through covered stent (3000) followed by balloon (2550) advancement into the center of the stent and balloon inflation (images d and e).

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When embodiments are referred to as “exemplary” or “preferred” this term is meant to indicate one example of the invention, and does not exclude other possible embodiments. When structures are identified as a means to perform a function, the identification is intended to include all structures which can perform the function specified. When structures of this invention are identified as being coupled together, such language should be interpreted broadly to include the structures being coupled directly together or coupled together through intervening structures. Such coupling could be permanent or temporary and either in a rigid fashion or in a fashion which allows pivoting, sliding or other relative motion while still providing some form of attachment, unless specifically restricted.

What is claimed is:

1: A radially expandable stent comprising:
a first end portion;
a second end portion;
a central portion between said first end portion and said second end portion; and
said central portion having a width perpendicular to a central axis of the stent that is narrower than a width of said first end portion and said second portion, after at least initial stent radial expansion.

2: The stent of claim 1 wherein said first end portion and said second end portion include a first collapsed diameter and a second expanded diameter, said second expanded diameter greater than said first collapsed diameter.

3: The stent of claim 2 wherein said first end portion and said second end portion are biased toward said second expanded diameter, such that when restraining forces are removed from said first end portion and said second end portion, said first end portion and second end portion automatically transition to said second expanded diameter.

4: The stent of claim 2 wherein said first end portion and said second end portion are biased toward said first collapsed diameter, but are able to be flexibly expanded to said second expanded diameter by application of radial forces thereto.

5: The stent of claim 4 wherein said central portion has greater resistance to radial expansion than said first end portion and said second end portion.

6: The stent of claim 5 wherein said central portion can be radially expanded to a second expanded diameter matching said second expanded diameter of said first end portion and second end portion when sufficient radial expansion forces are applied to said central portion.

7: The stent of claim 1 wherein a sheath surrounds said stent before deployment, said sheath sufficiently strong to hold said first end portion and said second end portion in a collapsed configuration.

8: The stent of claim 7 wherein said collapsed configuration includes said first end portion, said second end portion and said middle portion having similar diameters.

9: The stent of claim 8 wherein said stent includes an expanded configuration wherein said first end portion and said second end portion have similar diameters, and said middle portion has a smaller diameter than said end portions.

10: The stent of claim 1 wherein said stent includes a foraminous metal cage forming said first end portion, said second end portion and said middle portion.

11: The stent of claim 1 wherein said stent includes at least one non-porous flexible layer adjacent to said metal cage.

12: The stent of claim 1 wherein said flexible layer is on an inside surface of said metal cage.

13: The stent of claim 1 wherein a second flexible layer is provided on an outside surface of said metal cage.

14: The stent of claim 11 wherein said flexible layer includes ePTFE.

15: The stent of claim 14 wherein said flexible layer is sufficiently flexible to avoid rupture when said stent is expanded radially.

16: The stent of claim 1 wherein each of said end portions and said middle portion each have circular cross-sections along lengths thereof, and wherein said end portions include a conical tapering diameter portion, with smallest diameters adjacent to said central portion.

17: A method for providing a stenosis narrowing within a blood vessel, fistula, or graft, the method including the steps of:
locating an expandable stent within a sheath, the sheath holding the stent in a non-expanded form, the stent including a first end portion, a second end portion, and a central portion between the first end portion and the second end portion, the central portion having a width perpendicular to a central axis of the stent that is narrower than the first end portion and the second end portion, after at least initial stent radial expansion; and
retracting the sheath to leave the stent at an implantation location.

18: The method of claim 17 wherein said stent self-expands when said sheath is retracted during said retracting step.

19: The method of claim 17 including the further step of expanding the sheath by expanding a balloon within the stent, with an initial stent radial expansion which expands the first end portion and the second end portion to a greater diameter than the central portion.

20: The method of claim 19 including the further step of further expanding the sheath by further expanding the balloon within the stent to cause the central portion to be expanded to a similar diameter as said first end portion and said second portion.
